(19) United States
(12) Patent Application Publication
(10) Pub. No.: US 2009/0275529 A1
(43) Pub. Date: Nov. 5, 2009

(54) METHOD FOR IMPROVING CARDIOVASCULAR RISK PROFILE OF COX INHIBITORS

(76) Inventors: Allison B. Reiss, Forest Hill, NY (US); Steven Carsons, Syosset, NY (US)

Correspondence Address: OSTROLENK FABER GERB & SOFFEN, 1180 AVENUE OF THE AMERICAS, NEW YORK, NY, 100368403, US

(21) Appl. No.: 12/435,843

(22) Filed: May 5, 2009

Related U.S. Application Data
(60) Provisional application No. 61/050,499, filed on May 5, 2008.
(60) Provisional application No. 61/115,289, filed on Nov. 17, 2008.

(51) Int. Cl. A61K 31/7076; A61K 31/415; A61K 31/42; A61K 31/34; A61P 9/10

(52) U.S. Cl. 514/46; 514/406; 514/378; 514/473

(57) ABSTRACT

A method of reducing the increased risk of cardiovascular events attendant with the use of COX or selective COX-2 inhibitors including restoring disrupted cholesterol metabolic function engendered by the use of the COX or selective COX-2 inhibitors and reducing the production of foam cells caused thereby. The disrupted cholesterol metabolic function is restored and the production of foam cells is reduced by the step of administering an adenosine A2A receptor agonist having a threshold level of activity of 0.1 μM to a patient using the COX or COX-2 inhibitor. The adenosine A2A receptor agonist is administered orally in amounts sufficient to saturate the A2A receptor and at time intervals sufficient to maintain the restored cholesterol metabolic function.

### CROSS-REFERENCE TO RELATED APPLICATIONS

This application is a non-provisional application claiming priority from provisional application No. 61/050,499, filed May 5, 2008 and No. 61/115,289, filed Nov. 17, 2008, the disclosures of which are incorporated herein in their entireties, by reference thereto.

### FIELD OF THE INVENTION

This invention relates to methods for protecting the mammalian, including the human, cardiovascular system from the adverse effects of cyclooxygenase (COX) inhibitors including those of selective COX-2 inhibitors.

### BACKGROUND

The association of COX inhibitor NSAIDs (non-steroidal anti-inflammatory drugs), including COX-2 inhibitors, with elevated risk of myocardial infarction and stroke is a major public health concern. The worldwide withdrawal of the selective COX-2 inhibitor, rofecoxib (Vioxx®, Merck) because of adverse cardiovascular events, has complicated clinical decision making for the physician treating patients with pain and inflammation. Whether the benefits of COX-2 inhibitors (also referred to colloquially as “coxibs”) and other COX inhibiting NSAIDs outweigh their cardiovascular risks is the subject of an ongoing debate.

Selective inhibitors of COX-2 are highly effective anti-inflammatory and analgesic drugs that exert their action by preventing the formation of prostanoids. They are used clinically to relieve the symptoms of osteoarthritis and rheumatoid arthritis, and to treat dysmenorrhea.

In September 2004, rofecoxib was withdrawn from the market after clinical trials showed a significant increase in the incidence of adverse cardiovascular events, particularly fatal myocardial infarctions (heart attacks), in rofecoxib-treated subjects compared with placebo. Valdecoxib (Bextra®, Pfizer) was subsequently withdrawn from the market after trials found significantly increased risk of heart attack and stroke. It has become generally known that COX-2 inhibitors, as a class, significantly elevate heart attack and stroke incidence.

The precise mechanisms by which selective inhibitors of COX-2 predispose individuals to cardiac and cerebrovascular disease (stroke) have not been elucidated. Since COX-2-derived prostacyclins exert a cardioprotective effect, the actual increased risk may be related to a decrease in their expression. In fact, one hypothesis to explain the increased cardiovascular risk associated with COX-2 inhibition is the accompanying fall in prostacyclin levels, which may leave arteries more vulnerable to clotting. However, there is a “latent period” before emergence of cardiovascular risk, such as the 18 months prior to the noted increase in incidence of heart attack and stroke in patients treated with rofecoxib, in a Merck funded APPROVe study, that has not been satisfactorily explained or accounted for.

### SUMMARY

We have discovered that the use of COX inhibitor NSAIDs, including COX-2 inhibitors, has a disruptive effect on the normal cholesterol metabolic function, with cholesterol not being efficiently cleared from arterial walls. This increase in retained cholesterol was accompanied by increased production of detrimental lipid laden foam cells which attach to arterial walls leading to atherosclerosis and cardiovascular problems including heart attacks, and cerebrovascular problems (stroke). Since atherosoclerosis is a condition which worsens over time, the delay or “latent period” before emergence of cardiovascular risk is explainable.

The cholesterol metabolic function involves macrophage cytochrome P450 cholesterol 27-hydroxylase (hereinafter “27-OHase”) and other reverse cholesterol transport (RCT) proteins which counteract cholesterol accumulation by promoting cholesterol efflux. In addition, ATP-binding cassette transporter A1 (hereinafter “ABCA1”), an anti-atherogenic integral membrane protein is also a factor in the RCT process.

We discovered that COX inhibitor NSAIDs and selective COX-2 inhibitors reduce the expression of the RCT proteins 27-OHase and ABCA1 which results in disruption of cholesterol metabolism, to the extent that cholesterol efflux is reduced, cholesterol is retained and lipid laden foam cells are produced. The lipid laden foam cells become attached to arterial walls forming plaque which leads to the adverse cardiovascular condition of atherosclerosis, with increased risk of adverse cardiovascular events such as heart attacks and stroke. We concluded that COX and COX-2 inhibition is an atherogenic effect that may, therefore, contribute to heightened risk of development of atherosclerotic cardiovascular disease (ASCVD) associated with prolonged use of this drug class.

The present invention relates to a method of reducing the increased risk of cardiovascular and cerebrovascular problems and adverse events in mammals including humans, including those related to atherosclerosis, which may result from the use of selective COX-2 inhibitors or other COX inhibitor NSAIDs. The present invention also relates to a pharmaceutical composition useful in such method. As used herein the term “cardiovascular events” means adverse events such as myocardial infarctions and strokes, attributable to ASCVD and/or disruption of the cholesterol metabolism.

The method comprises restoring disrupted cholesterol metabolic function engendered by the use of COX or selective COX-2 inhibitors and reducing the production of lipid laden foam cells caused by the use of COX or selective COX-2 inhibitors. The disrupted cholesterol metabolic function is restored and the production of lipid laden foam cells is reduced by administering a pharmaceutically acceptable adenosine A2A receptor agonist having a threshold level of activity or receptor affinity achieved beginning at a concentration of 0.1 μM and optimal at 1 μM to a patient using the COX or selective COX-2 inhibitor. The adenosine A2A receptor agonist is administered in amounts sufficient to saturate the A2A receptor and at time intervals sufficient to maintain the restored cholesterol metabolic function. As used herein, “administration” of the adenosine A2A receptor agonist also includes administration of a material which effectively results in production in situ of an adenosine A2A receptor agonist.

The method reduces the increased risk of adverse atherosclerotic cardiovascular and cerebrovascular conditions in a human resulting from the use by the human of a COX or selective COX-2 inhibitor which disrupts a normal cholesterol metabolic function and causes production of lipid laden foam cells. The method comprises restoring disrupted cholesterol metabolic function engendered by COX or selective COX-2 inhibitor, and reducing the production of lipid laden foam cells caused by the use of the COX or COX-2 inhibitor.

The method also ameliorates atherogenesis in a mammal including human, by administration of an adenosine A2A receptor agonist to the mammal in need thereof in an amount substantially only sufficient to saturate the A2A receptor of the human, wherein atherogenesis in the subject is thereby reduced or prevented.

The pharmaceutical composition comprises a pharmaceutically acceptable COX or selective COX-2 inhibitor and a pharmaceutically acceptable A2A receptor agonist, with the A2A receptor agonist being present in an amount sufficient to restore cholesterol metabolic function disrupted by the COX or selective COX-2 inhibitor. The pharmaceutical composition of the present invention also comprises a COX or COX-2 inhibitor admixed with an adenosine A2A receptor agonist or a material which effectively results in production in situ of the adenosine A2A receptor agonist. The pharmaceutical composition comprises an analgesic or antiinflammatory effective amount of the COX or COX-2 inhibitor and includes, but is not limited to the standard pharmaceutical dose of the COX or COX-2 inhibitor (e.g., standard doses of commercial Celebrex COX-2 inhibitor are 50, 100, 200 and 400 mg) and an amount of the adenosine A2A receptor agonist sufficient to saturate the A2A receptor. The material (e.g., methotrexate (MTX) which mediates production of the A2A receptor agonist, adenosine in situ) which effectively results in the production of the adenosine A2A receptor agonist should be present in an amount which effectively results in the adenosine A2A receptor agonist in amounts sufficient to saturate the A2A receptor. The pharmaceutical composition may further include carriers, excipients and other pharmaceutically acceptable materials as normally used in pharmaceutical compositions of the COX or COX-2 inhibitors.

### BRIEF DESCRIPTION OF THE DRAWINGS

FIGS. 1a and 1b are bar graphs showing the reduction effect of celecoxib (Celebrex® Pfizer), selective COX-2 inhibitor at different dose levels, on the gene expression (FIG. 1a) and protein expression (FIG. 1b) of ABCA1, respectively.

FIGS. 2a and 2b are bar graphs showing the extent of reduction of 27-OHase (FIG. 2a) and ABCA1 (FIG. 2b) respectively, caused by the selective COX-2 inhibitor, NS-398 with different dose levels.

FIGS. 3a-3d are bar graphs showing the effect of adenosine A2A receptor agonists on 27-OHase levels with combinations of different A2A receptor agonists, and COX-2 inhibitors and the effect of an A2A antagonist on the combinations (FIGS. 3b and 3c).

FIGS. 4a and 4b are bar graphs showing that the ABCA1 level effects of A2A agonist CGS-21680 and A2A agonist, methotrexate (MTX), respectively are reduced by an adenosine A2A receptor antagonist.

FIG. 5 is a bar graph showing the effects on 27-OHase levels of NS-398 and MTX at different concentration levels of MTX.

FIGS. 6a and 6b are bar graphs of the effects on 27-OHase and ABCA1 levels respectively of MTX, an A2A antagonist, atherosclerosis promoting cytokine, IFN-γ, and NS-398 and combinations thereof.

### DETAILED DESCRIPTION OF THE EMBODIMENTS

We found that adenosine A2A receptor agonists, including those currently used in development of asthma and inflammation treatments as well as in myocardial imaging, provide the unexpected effect of substantially reversing the disruption of the RCT process engendered by COX and selective COX-2 inhibitors by increasing the expression of the RCT proteins. The A2A receptor agonists further inhibit production of detrimental lipid laden foam cells which attach to arterial walls resulting plaque deposits and an atherosclerotic condition.

The amount of the A2A receptor agonist effective in reversing the disruption of the RCT process is minimal and only a fraction of the dosage required in treating asthma, inflammation or for imaging purposes, with the requirement being that the A2A receptor agonist has an A2A receptor agonism activity level in the nanomolar range and is present in sufficient amounts to saturate the high affinity A2A receptor sites. Saturation analysis with [3H]NECA gave a KD-value of 20 nM for the human A2A adenosine receptor.

The activity half life of the specific A2A receptor agonist is determinative of the concurrent dosing thereof with respect to concurrent use of the COX or COX-2 inhibitor. Accordingly, the activity half life of the specific A2A receptor agonist will affect the required frequency of dosing. Dosing with the A2A receptor agonist is only required at time intervals necessary to maintain A2A receptor site saturation. The terminal half-life in rats after an IV dose of CGS-21680 (0.3 mg/kg) is very short (19±4 min), but newer drugs have longer half lives than their predecessors.

Possible side effects with long term usage of the A2A agonists concurrent to dosing use of COX and COX-2 inhibitors are minimized to acceptable risk levels with the small amounts necessary to maintain A2A receptor saturation as well as the extended time periods (depending on A2A agonist half life) required to maintain receptor saturation.

Although adenosine A2A agonists often have activity at other sites, A2A agonists which are specific to the A2A receptor are likely to have enhanced efficiency and have minimized side effects which may occur with activation of other adenosine receptors not involved in restoration of the cholesterol metabolic function. Currently, in addition to the existing A2A agonists, with activity at other receptors, new A2A specific agonists have been developed and are at varying stages of approval for use in humans. Except as otherwise indicated the term “A2A receptor agonist” includes materials which function to mediate production in situ of A2A receptor agonists.

The presently known adenosine receptor subtypes are A1, A2A, A2B and A3 with only activation of the A2A receptor sites being effective in restoring RCT in accordance with the present invention. Adenosine itself however has too short a half life to provide any effectiveness with direct administration. The following are suitable materials effective as A2A receptor agonists in restoring RCT in accordance with the present invention. Materials such as methotrexate (MTX), which is approved for use in humans and is used in cancer treatment in large doses and as the treatment of choice for rheumatoid arthritis (RA) in smaller doses, though not itself an A2A agonist, mediates adenosine release which has activity as an A2A agonist, and thus has activity as an A2A agonist as used herein. Experimentally available CGS-21680 (though not approved for use in humans) is primarily an A2A agonist (though with some activity with other receptors) and is an effective material for use in restoring cholesterol metabolic function.

Other current A2A receptor agonists in development and/or approved for use in humans and which are useful in the present invention include Binodenoson (CorVue™), MRE-0094, UK-371,104, ATL 313, Regadenoson (Lexiscan™), Apadenoson, APEC and 2HE-NECA.

The discussion below describes details of the experimental methods, materials and results used herein.

The effect of COX inhibition on the expression of several proteins involved in cholesterol efflux in THP-1 human monocytes and macrophages was evaluated. We found that pharmacological inhibition of COX-1 and/or COX-2 greatly reduced 27-OHase and ABCA1 expression and that this led to an increase of foam cell formation and an atherosclerotic condition.

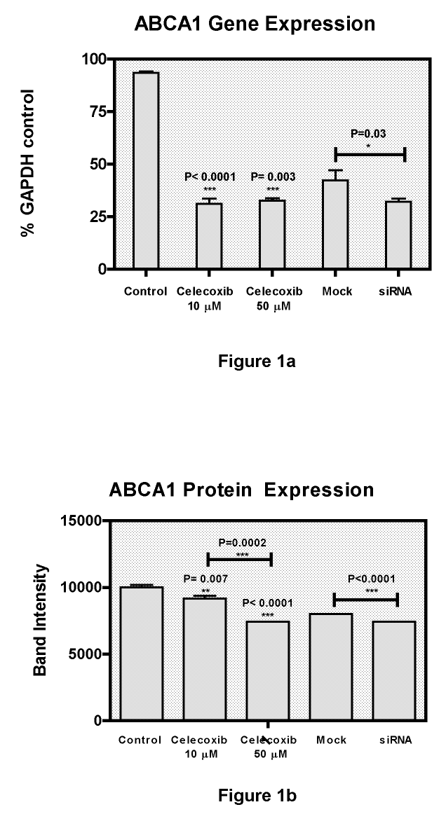

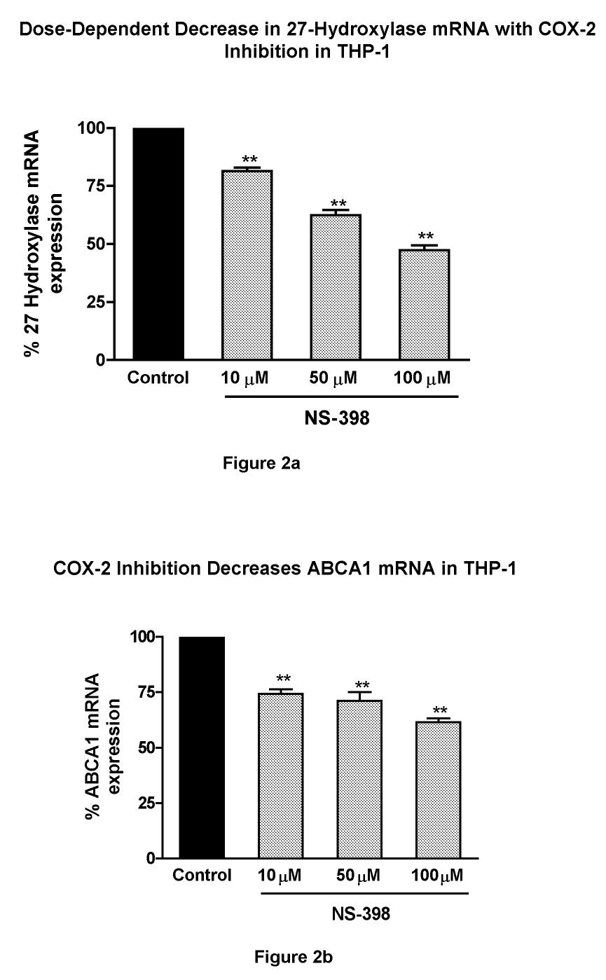

FIGS. 1a and 1b show the reduction of ABCA1 gene expression and protein expression respectively with the use of celecoxib at dose concentrations of 10 μM and 50 μM, which explains, at least in part, the propensity toward atherogenesis with COX inhibition. The COX-2 selective inhibitor NS-398 markedly diminished 27-OHase and ABCA1 message and protein in TUP-1 monocytes and macrophages in a dose dependent manner as shown in FIGS. 2a and 2b respectively. NS-398 reduced 27-OHase mRNA in TUP-1 to 62.4±2.2% of control (50 μM, n=3, p<0.001). TUP-1 macrophages treated with NS-398 also showed greater vulnerability to form lipid-laden foam cells compared to untreated cells, with TUP-1 macrophages showing a significant increase in foam cell transformation in the presence of NS-398 compared to control (42.7±6.6 vs. 20.1±3.4%, p=0.04).

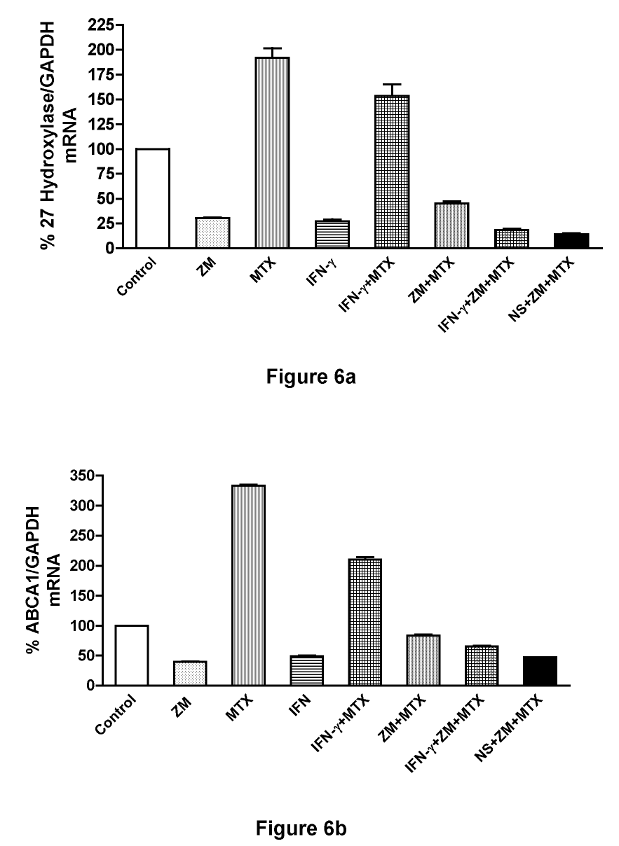

We also discovered that immune reactants interfere with cellular defense against cholesterol overload by diminishing expression of the two proteins responsible for reverse transport of cholesterol out of the cell to the circulation for ultimate excretion: cholesterol 27-OHase and ABCA1, with the atherosclerosis-promoting cytokine IFN-γ decreasing 27-OHase and ABCA1 message and protein expression in THP-1 human monocytes/macrophages as shown in FIGS. 6a and 6b respectively.

Further, IFN-γ-treated TUP-1 macrophages formed foam cells more rapidly and in greater proportion than untreated control cells. Inhibition of cyclooxygenase (COX) in THP-1 monocytes/macrophages acts in a pro-atherogenic manner by dose-dependently decreasing 27-OHase and ABCA1. TUP-1 macrophages showed a significant increase in foam cell transformation in the presence of the COX-2 selective inhibitor NS-398 compared to control. This indicated that compromise or disruption of reverse cholesterol transport (RCT) contributes to the known increase in cardiovascular risk in patients treated with COX-2 inhibitors.

We discovered that these proteins can be upregulated toward normal cholesterol metabolic function via activation of the adenosine A2A receptor such as with specific A2A agonists including CGS-21680 and MRE-0094. Ligation of the A2A receptor also inhibited macrophage foam cell transformation under cholesterol loading conditions. We found that methotrexate (MTX) also modulates cholesterol metabolism and vulnerability to foam cell formation and counteracts propensity toward cholesterol overload in TUP-1 monocytes/macrophages exposed to IFN-γ or selective COX-2 inhibition. MTX provides this protection from atherosclerotic cardiovascular disease (ASCVD) by adenosine release. MTX is not an adenosine A2A receptor agonist but is a source of adenosine which has A2A receptor agonist activity and MTX is accordingly an A2A receptor agonist within the scope of the present invention.

As discussed in greater detail below, we established that COX-2 inhibitors, as a class, disrupt the known cholesterol metabolic function of the RCT process, with resultant cholesterol retention and production of lipid laden foam cells. FIGS. 1a and 1b show the effect of celecoxib in reducing ABCA1 gene and protein expression as compared to a control. FIGS. 2a and 2b show the dose dependent continued decrease of 27-OHase in TUP-1 (FIG. 2a) and the dose dependent continued decrease of ABCA1 in TUP-1 (FIG. 2b) of the COX-2 inhibitor NS-398.

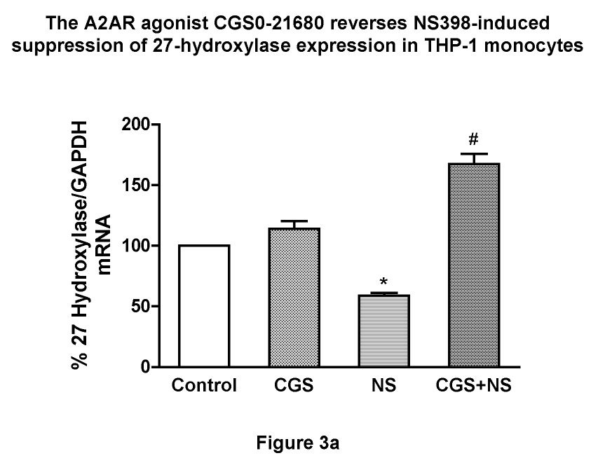

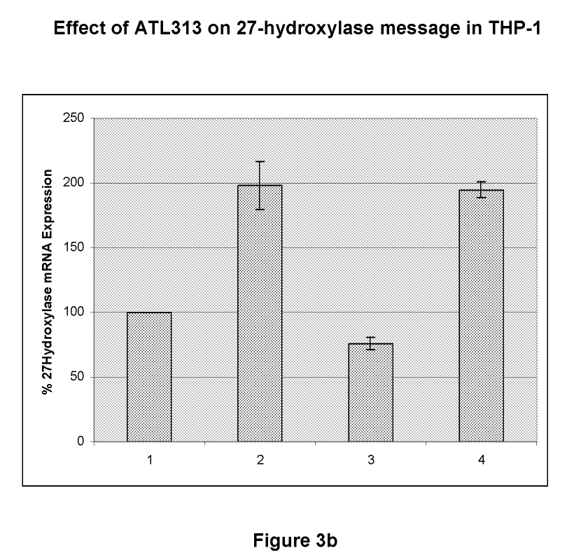

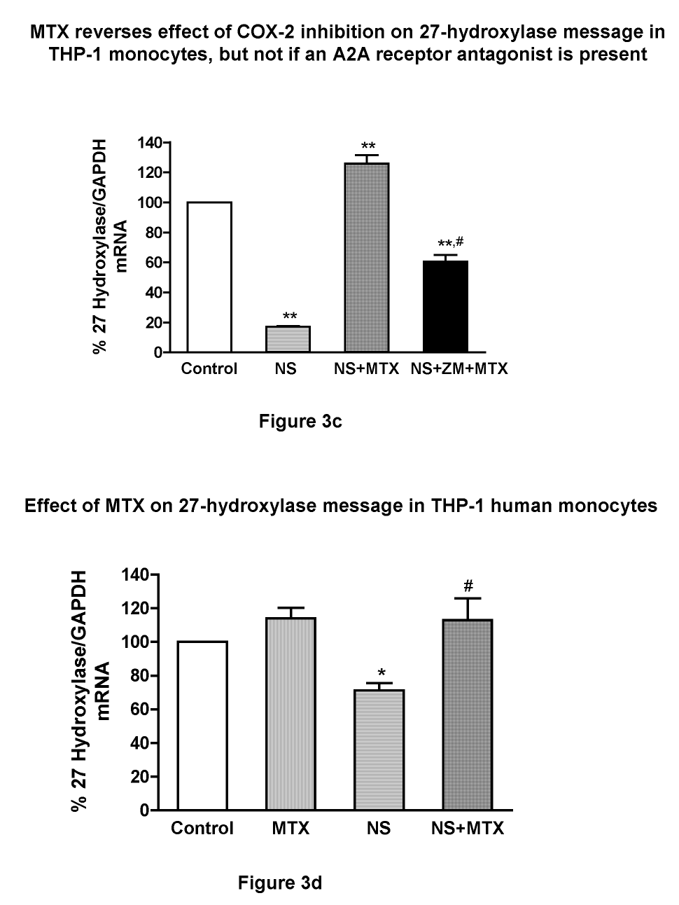

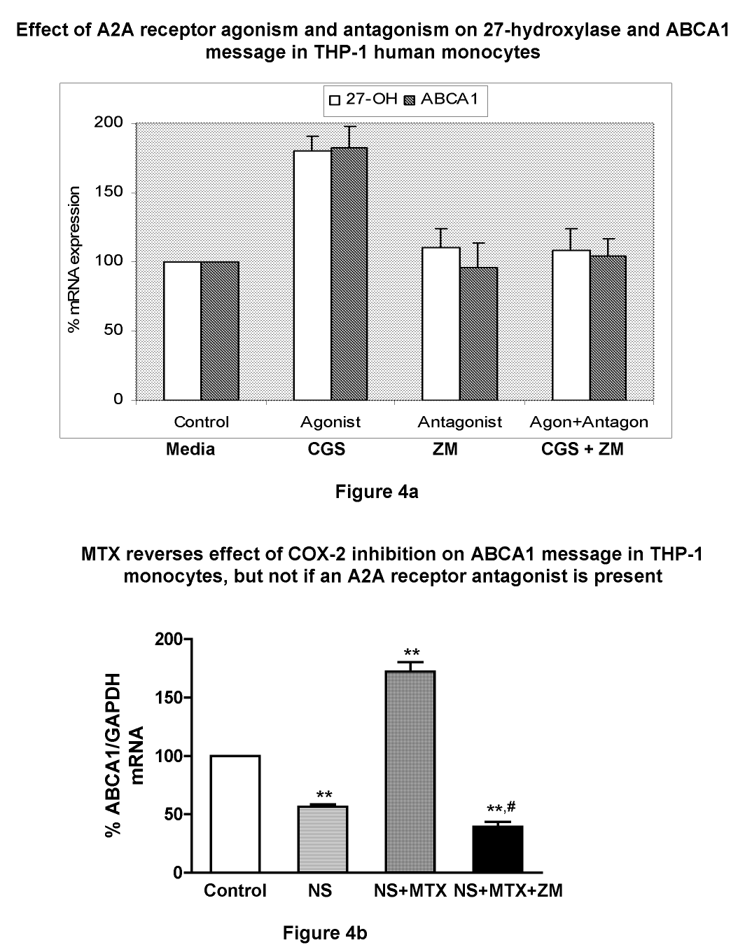

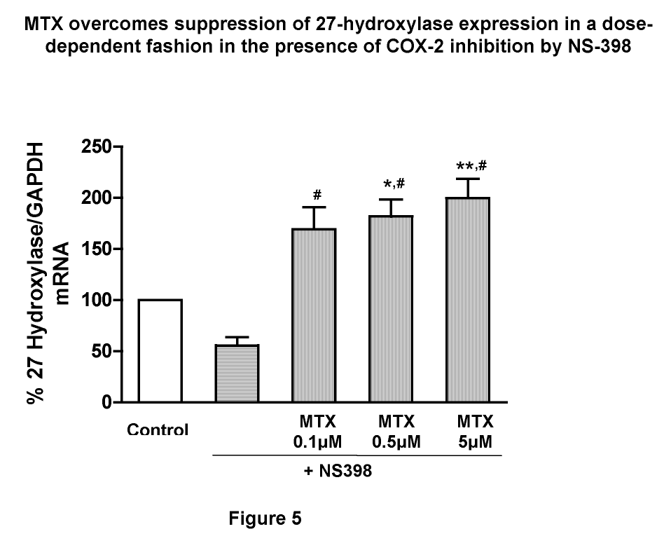

Further, we established that adenosine A2A receptor agonists restore the cholesterol metabolic function. FIG. 3a shows restoration of 27-OHase levels in TUP-1 by use of A2A agonist CGS-21680. FIGS. 3c, 3d and 5 show a similar restoration of 27-OHase levels with use of MTX from levels reduced by NS-398. FIG. 4b shows restoration by MTX if ABCA1 message originally reduced by MS-398.

We also demonstrated that the activity of the adenosine A2A receptors is responsible for controlling the disruption and restoration of the cholesterol metabolic function. FIG. 3b shows the effect of A2A agonist ATL313 in generally raising 27-OHase levels in TUP-1. ATL313 increases 27-OHase message in TUP-1 macrophages and this is reversed by ZM-241385 (ZM). THP-1 macrophages were exposed to the following conditions represented by the four bars from left to right: 1) 0.5 nM DMSO [vehicle] 18 hr; 2) ATL313 10 nM 18 hr; 3) ZM 10⁻⁵M 1 hr pre-incubation followed by ATL313 10 nM for a further 18 hr. 4) CGS-21680 10⁻⁵M 18 hr. Total RNA was extracted and evaluated for 27-OHase mRNA by QRT-PCR. Amplification of GAPDH message was used as an internal control.

The 27-OHase and ABCA1 levels were determined with various combinations of selective COX-2 inhibitors, and adenosine A2A receptor agonists (since MTX mediates production of adenosine which has A2A receptor agonist properties, MTX is considered as an A2A agonist herein for simplification).

NS-398 (an available selective COX-2 inhibitor) was used in the testing as well as celecoxib (the only commercially available selective COX-2 in the US), both of which exhibited cholesterol metabolic function disruption, as discussed. Specific A2A receptor agonists MTX, CGS-21680, MRE-0094 and ATL313 all exhibited restoration of the cholesterol metabolic function. Dispositive confirmation of the sole implication of the A2A receptor activity in the restoration was made with the use of known A2A antagonist ZM-241385 to block A2A receptor sites and with concomitant prevention of the restoration of the cholesterol metabolic function by the A2A agonists used, as shown in FIGS. 3b-3d, 4a, 4b, 6a and 6b.

Since MTX is known to affect both adenosine release and cardiovascular risk, MTX was used to determine if it modulates cholesterol metabolism and vulnerability to foam cell formation. We discovered that MTX treatment counteracts propensity toward cholesterol overload in TUP-1 monocytes/macrophages exposed to IFN-γ or selective COX-2 inhibition. MTX thus provided protection from atherosclerotic cardiovascular disease (ASCVD) by increasing expression of anti-atherogenic molecules involved in cholesterol efflux.

MTX increases 27-OHase in healthy monocytes isolated from peripheral blood, in addition to use of a monocytic cell line. The peripheral monocytes were primary cells taken directly from the human body and there is evidence of direct physiologic effect by using them.

Celecoxib or Celebrex (registered trademark of Pfizer Inc.) acts directly on cultured TUP-1 monocytes to decrease ABCA1. This indicates that results with the COX-2 inhibitor NS-398 are generalizable to other COX inhibitors that are used in people. A COX-2 silencing study showed that turning off the COX-2 gene by non-pharmacologic methods works as well.

Celecoxib increases foam cell formation in TUP-1 macrophages so not only does celecoxib affect gene expression, it directly impairs the ability of the cell to rid itself of cholesterol and defend itself against lipid overload (the key process that initiates atherosclerotic plaque formation) via a pathway involving adenosine release.

In addition to MTX, A2A agonist CGS-21680 was also found to increase 27-OHase and ABCA1 Message in Murine and Human Monocytes.

A number of different highly receptor-specific agonists and antagonists of adenosine receptors have been developed that either mimic or block the effects of adenosine. We found that the selective A2A receptor agonist, 2-(4-(2-carboxyethyl)phenethylamino)-5′-N-ethylcarboxamidoadenosine (CGS-21680), at concentrations of 10⁻⁵M (3 hours, 37° C., 5% CO2), increases 27-OHase mRNA expression in Balb/C murine macrophages by 47±6.2% (n=3 per group, student t-test, p<0.002). This is believed to be the first demonstration that an endogenous agent upregulates 27-OHase mRNA expression. Based on the upregulation of 27-OHase in murine cells upon exposure to CGS-21680, the same effect was established in human monocytoid cells. In THP-1 cells, ABCA1 mRNA increased in concert with 27-OHase. THP-1 cells were exposed to CGS-21680 in the presence and absence of the A2A receptor antagonist ZM-241385 and it was found that both 27-OHase and ABCA 1 message levels failed to rise when the antagonist was present, as shown in FIG. 4a.

In THP-1 cells, CGS-21680 increased 27-OHase message in a dose dependent fashion by as much as 1.8-fold. A more selective A2A receptor agonist MRE-0094 also increased 27-OHase (2-fold) and ABCA1 (1.8-fold) mRNA expression in THP-1 and the A2AR antagonist ZM-241385 reversed the effect of MRE-0094 on both these messages.

Until recently, major obstacles to the clinical use of A2A receptor agonists included necessity of intravenous administration and short half-life of compounds. Newly developed long acting, orally active A2A receptor ligands include the promising new agonist ATL313. Consistent with the results with CGS-21680, it was found that ATL313 exerts powerful antiatherogenic effects on TUP-1 macrophages in culture. ATL313 increases 27-OHase as shown in FIG. 3b and ABCA1 mRNA and protein in TUP-1 macrophages and these effects are blocked by ZM-241385.

In order to prove that only the A2A receptor is specifically implicated in the RCT restoration, the below procedures were conducted.

CGS-21680 was used as an activator of the adenosine A2A receptor to show reversal of the NS-398 effect on 27-OHase level. This does not however permit the conclusion that ligation of the A2A receptor is responsible for reversing COX-2 effects because CGS-21680 may have other actions such as binding to the A1 receptor.

In a crucial step further to confirm that the effect is A2A-mediated, loss of the effect occurred when the A2A receptor was blocked by adding A2A antagonist, ZM-241385, which blocked the A2A receptor so that it cannot be turned on. When the COX-2 inhibitor was added with MTX (which causes adenosine release), MTX did not stop the COX-2 inhibitor from decreasing 27-OHase and ABCA1 in the presence of ZM-241385. This conclusively indicated that MTX or A2A agonists generally cannot work without access to the A2A receptor.

Oral dosing and oral active drugs that can be administered to humans—MTX and ATL313 were tested as set forth below.

Though the effect of the A2A receptor agonist CGS-21680 was examined, it should be noted that use of CGS-21680 may be inconvenient because CGS-21680 must be infused and at present, is only used experimentally in animals and not in humans. However, both methotrexate and the A2A specific drug ATL313 (developed by Adenosine Therapeutics) are orally active and there are currently a series of ATL adenosine A2A agonists that are either in clinical trials or will be shortly for human use in a variety of disorders. Dosage levels in mice of ATL313 were initially at 5 micrograms/kg every six hours in a murine sepsis model. Subsequent dosage levels were at 30 micrograms/kg/day with the ATL313 being added to their food. A typical MTX regimen for rheumatoid arthritis in humans would be to start MTX at 10 mg/week with dose escalation to 20-25 mg/week by week 8.

Decreased Foam Cell Formation was found with MTX as the A2A agonist and NS-398 as the COX-2 inhibitor tests were conducted as set forth below.

Lipid overloaded macrophage-derived foam cells play a fundamental role in all stages of atherosclerosis. A direct physiologic link was shown between the exposure of macrophages to NS-398 and the ability of the cells to defend against cholesterol overload. A series of experiments were performed which demonstrated that foam cell formation from TUP-1 macrophages in the presence of acetylated LDL increases dramatically upon exposure to NS-398 and this effect is mitigated by MTX. Since the mitigation by MTX is due to A2A receptor activation, it was found that ZM-241385 prevents MTX from decreasing NS-398-induced foam cell formation. The effect of MTX and NS-398 on cholesterol 27-OHase and ABCA1 correlate to a high degree with the physiologic phenomena seen in lipid-exposed TUP-1 macrophages.

Methods: Materials and Sources

This section outlines the material, including biologic materials used throughout all the following experiments in showing: a) cholesterol metabolism disruption by COX inhibitors; b) restoration of cholesterol metabolism as measured by 27-OHase and ABCA1 levels, by use of A2A agonists (including MTX which produces an A2A agonist in situ); and c) that only the A2A receptor is involved in both disruption and restoration of the cholesterol metabolism as measure by 27-OHase and ABCA1 levels.

Cells and reagents: TUP-1 monocytes were obtained from ATCC (Manassas, Va.). Oil red O and OptiPrep Density Gradient Media were purchased from Sigma (St. Louis. Mo.). Trizol reagent was purchased from Invitrogen (Grand Island, N.Y.). All reverse transcription-Polymerase chain reaction (RT-PCR) reagents were purchased from Applied Biosystems, Roche. Recombinant human IFN-γ was purchased from R&D Systems (Minneapolis, Minn.). NS-398 was purchased from RBI-Sigma, Natick, Mass. MTX was purchased from Bedford Laboratories, Bedford Ohio. Acetylated LDL was purchased from Intracel (Issaquah, Wash.). Anti-cholesterol 27-OHase antibody is an affinity-purified rabbit polyclonal anti-peptide antibody raised against residues 15-28 of the cholesterol 27-OHase protein.

Preparation of Biologic Materials

Cell culture: TUP-1 monocytes were grown at 37° C. in a 5% CO₂ atmosphere to a density of 10⁶ cells/ml. Growth medium for TUP-1 cells was RPMI 1640 (GIBCO BRL, Grand Island, N.Y.) supplemented with 10% Fetal Bovine Serum (FBS) (GIBCO BRL), 50 units/ml penicillin, and 50 units/ml streptomycin. To facilitate differentiation into macrophages, TUP-1 monocytes (10⁶ cells/ml) in 12 well plates were treated with 100 nM PMA (Sigma) for 4 days at 37° C.

PBMC (peripheral blood mononuclear cell) isolation: Blood from healthy donors was collected in EDTA treated tubes, pooled and kept at 4° C. The pooled blood was adjusted to a density of 1.120 g/ml with the addition of OptiPrep Density Gradient Media (Sigma) according to the manufacturer's instructions. The blood was then overlaid with a 1.074 g/ml density solution composed of complete RPMI containing 10% FBS and OptiPrep media. A layer of complete RPMI containing 10% FBS was then overlaid on top to prevent monocytes from sticking to the plastic tube. The blood was centrifuged at 750 g for 30 minutes at 4° C. After centrifugation, the monocyte interphase was collected from between the 1.074 g/ml and RPMI layer. The collected cells were diluted with 2 volumes of complete RPMI and harvested by centrifugation. The pellet was re-suspended in complete RPMI. The monocytes were counted by hemocytometer and plated at a density of 2×10⁶ cells/well in a 6-well plate.

Methods: COX Inhibitors with Adenosine A2A Agonists, Antagonists

THP-1 cells: When THP-1 cells had reached 10⁶ cells/ml, media was aspirated and cells were rinsed twice with Dulbecco's Phosphate Buffered Saline (DPBS) without calcium and magnesium. The monocytes were then incubated for 24-48 hours in six well plates, (37° C., 5% CO₂) under the following conditions: a) RPMI control; b) RPMI containing 5 mM MTX; c) RPMI containing NS-398 (50 μM); d) RPMI containing NS-398 (50 μM) and MTX (increasing doses of 0.1 μM, 0.5 μM and 5 μM); e) RPMI containing IFN-γ (500 U/ml); f) RPMI containing IFN-γ (500 U/ml) and 5 μM MTX.

TUP-1 macrophages were exposed to the following conditions: a) RPMI control; b) RPMI containing ZM-241385 (10 μM); c) RPMI containing MTX (5 μM); d) RPMI containing IFN-γ (500 U/ml); e) RPMI containing IFN-γ (500 U/ml) and 5 μM MTX; f) RPMI containing ZM-241385 (10 μM) and MTX (5 μM); g) RPMI containing IFN-γ (500 U/ml), ZM-241385 (10 μM) and MTX (5 μM); h) RPMI containing NS-398 (50 μM), ZM-241385 (10 μM) and MTX (5 μM).

Immediately after the incubation period, the cells were collected and centrifuged at 1500 RPM at room temperature, media was aspirated and cell protein and RNA were isolated. FIGS. 6a and 6b graphically depict the OHase and ABCA1 levels obtained.

PBMC: PBMC were incubated for 18 h in RPMI with 10% FBS with and without the addition of MTX at a concentration of 5 μM. Cells were collected and RNA isolated.

Other experimental conditions of TUP-1 and PBMC included the following parameters and conditions: when THP-1 monocytes had reached 10⁶ cells/ml, media was aspirated and cells were rinsed twice with Dulbecco's Phosphate Buffered Saline (DPBS) without calcium and magnesium. The monocytes were then incubated in six well plates, (37° C., 5% CO₂) under the following conditions: a) RPMI control; b) 10 μM NS-398 (18 h); c) 50 μM NS-398 (18 h); d) 10 μM celecoxib (18 hours); e) 50 μM celecoxib (18 h); f) IFN-γ (500 U/ml); g) 50 nM siRNA (24 h); h) 50 nM mock siRNA (24 h).

TUP-1 macrophages were cholesterol-loaded with 50 μg/ml acetylated LDL and incubated an additional 48 hours prior to oil red O staining. Immediately after the incubation period, the cells were collected and centrifuged at 1500 RPM at room temperature, media was aspirated and cell protein and RNA were isolated.

PBMC were incubated for 18 hours in RPMI with 10% FBS with and without the addition of celecoxib at a concentration of 50 μM. Cells were collected and RNA isolated.

Results

RNA isolation and quantitation: RNA was isolated using 1 ml Trizol reagent per 10⁶ cells and dissolved in nuclease-free water. The quantity of total RNA from each condition was measured by absorption at 260 and 280 wavelengths using quartz cuvettes by ultraviolet spectrophotometry (Hitachi U2010 spectrophotometer).

RT-PCR analysis of 27-OHase: 27-OHase and ABCA1 mRNA were quantitated by real-time PCR. cDNA was copied from 5 μg of total RNA using M-MLV reverse transcriptase primed with oligo dT. Equal amounts of cDNA were taken from each RT reaction mixture for PCR amplification using cholesterol 27-OHase-specific primers or ABCA1 specific primers as well as glyceraldehyde-3-phosphate dehydrogenase (GAPDH) control primers. The cholesterol 27-OHase-specific primers span a 311 base-pair sequence encompassing nucleotides 491-802 of the human cholesterol 27-OHase cDNA (24). ABCA1 primers yield a 234 BP amplified fragment. Real-time PCR analysis was performed using the SYBR Green PCR Reagents Kit (Applied Biosystems) with a Stratagene MX3005P QPCR System.

PCR was performed using techniques as follows. Each PCR reaction contained 2.5 μl of the 10 × fluorescent green buffer, 3 μl of 25 mM MgCl2, 2 μl dNTP mix (2500 μM dCTP, 2500 μM dGTP, 2500 μM dATP, and 5000 μM dUTP), 0.15 μl polymerase (5 U/μl; AmpliTaq Gold; Applied Biosystems), 0.25 μl uracil-N-glycosylase (1 U/μlUNG; AmpErase; Applied Biosystems), 0.5 μl of the forward and reverse primers (10 μM concentration), 4 μl cDNA, and water to a final volume of 25 μl. The thermal cycling parameters were as follows: 5 minutes at 95° C. to activate the polymerase (AmpliTaq Gold; Applied Biosystems), followed by 45 cycles of 30 seconds at 95° C. and 45 seconds at 58° C. then 45 seconds at 72° C. Each reaction was done in triplicate.

The amounts of PCR products were estimated, using software provided by the manufacturer (Stratagene). After completion of PCR cycles, the reactions were heat denatured over a 35° C. temperature gradient from 60° C. to 95° C. To correct for differences in cDNA load among samples, the target PCRs were normalized to a reference PCR involving the endogenous housekeeping genes glyceraldehyde-3-phosphate dehydrogenase (GAPDH) and β-actin. Nontemplate controls were included for each primer pair to check for significant levels of any contaminants. Fluorescence emission spectra were monitored and analyzed. PCR products were measured by the threshold cycles (CT), at which specific fluorescence becomes detectable. The CT was used for kinetic analysis and was proportional to the initial number of target quantity copies in the sample. A melting curve analysis was performed to assess the specificity of the amplified PCR products. The quantity of the samples was calculated after the CTs of the serial dilutions were compared with a control. QRT-PCR standards were prepared by making 1:10 serial dilutions of a purified PCR product.

Western Blots: Total cell lysates were prepared for Western immunoblotting using RIPA lysis buffer (98% PBS, 1% Igepal CA-630, 0.5% sodium deoxycholate, 0.1% sodium dodecyl sulfate [SDS]). 100 μl of RIPA lysis buffer and 10 μl of protease inhibitor cocktail (Sigma) were added to the cell pellet from each condition and incubated on ice for 35 minutes with vortexing every 5 min. Supernatants were collected after centrifuging at 10,000 g at 4° C. for 10 minutes using an Eppendorf 5415C centrifuge. The quantity of protein in each supernatant was measured by absorption at 560 nm using a Hitachi U2010 spectrophotometer.

Total cell lysate was used for Western blots. Protein samples (20 kg/lane) were boiled for 5 minutes, loaded onto a 10% polyacrylamide gel, electrophoresed for 1.5 hr at 100V then transferred to a nitrocellulose membrane in a semi-dry transblot apparatus for 1 hour at 100V. The nitrocellulose membrane was blocked for 4 hours at 4° C. in blocking solution (3% nonfat dry milk dissolved in 1×Tween20-tris-buffered saline [TTBS]) then immersed in a 1:300 dilution of primary antibody (18.7 μg/ml) in blocking solution overnight at 4° C. The primary antibody is an affinity-purified rabbit polyclonal anti-peptide antibody raised against residues 15-28 of the cholesterol 27-OHase protein. The following day, the membrane was washed 5 times in TTBS for 5 minutess per wash then incubated at room temperature in a 1:3000 dilution of ECL donkey anti-rabbit IgG Horseradish peroxidase-linked species-specific whole antibody (Amersham Biosciences, product Code NA934). The five washes in TTBS were repeated, then the immunoreactive protein was detected using ECL western blotting detection reagent (Amersham Biosciences, Cat No RPN2106) and film development in SRX-101A (Konica Minolta).

As a control, on the same transferred membrane, beta-actin was detected using mouse anti-beta-actin (diluted in 1:1000, from abCam, product Code: ab6276) and ECL sheep anti-mouse-IgG Horseradish peroxidase-linked species-specific whole antibody (diluted in 1:2000, from Amersham Biosciences, product Code NA931) and all other similar steps as above.

ABCA1 Detection: For ABCA1 detection, macrophage cell lysates were electrophoresed for 1.5 hr at 100V (10% polyacrylamide gel), then transferred to a nitrocellulose membrane. The membrane was blocked for 4 hours at 4° C. in blocking solution then incubated overnight at 4° C. in a 1:200 dilution of rabbit anti-ABCA1antibody (Santa Cruz Biotechnology). The following day, the membrane was washed 5 times in TTBS for 5 mins per wash then incubated at room temperature in a 1:5000 dilution of ECL donkey anti-rabbit IgG Horseradish peroxidase-linked species-specific whole antibody. Development proceeded as described above for the 27-OHase antibody.

Foam Cell Analysis: TUP-1 human monocytes (10⁶ cells/ml) in 12 well plates were treated with 100 nM PMA (Sigma) for 4 days at 37° C. to facilitate differentiation into macrophages. The differentiated macrophages were washed three times with phosphate-buffered saline (PBS) and further incubated in RPMI (37° C., 5% CO2) for 48 hr under the following five conditions: a) acetylated LDL (50 μg/ml); b) acetylated LDL (50 μg/ml) and IFN-γ (500 U/ml); c) acetylated LDL (50 μg/ml) and IFN-γ neutralizing antibody (1.2 μg/ml); d) acetylated LDL (50 μg/ml), IFN-γ (500 U/ml) and IFN-γ neutralizing antibody (1.2 μg/ml); e) acetylated LDL (50 μg/ml) and IFN-γ receptor antibody (125 ng/ml); f) acetylated LDL (50 μg/ml), IFN-γ receptor antibody (125 ng/ml) and IFN-γ (500 U/ml).

Immediately following incubation, media was aspirated and cells were fixed in the same 12 well plates used for incubation, with 4% paraformaldehyde in water, for 2-4 min. Cells were stained with 0.2% Oil-Red-O in methanol for 1-3 min. Cells were observed via light microscope (Axiovert 25-Zeiss) with 100× magnification and then photographed using a Kodak DC 290 Zoom Digital Camera. The number of foam cells formed in each condition were calculated manually and presented as percentage foam cell formation.

Data analysis: Statistical analysis were performed using GraphPad version 4.02 (GraphPad, San Diego, Calif.). All data were analyzed by one-way ANOVA and pairwise multiple comparisions were made between control and treatment conditions using Bonferroni's method.

Additional Experiments: TIP-1

The TUP-1 human monocyte-macrophage cell line was purchased from the American Type Culture Collection (ATCC, Manassas, Va.). TUP-1 cells were grown at 37° C. in a 5% CO2 atmosphere in the monocytic form in suspension in RPMI1640 supplemented with 10% FBS, 50 U/ml penicillin, and 50 U/ml streptomycin.

When TUP-1 monocytes reached a density of 1×106 cells per ml, they were rinsed twice with phosphate-buffered saline (PBS) without calcium and magnesium, then incubated in six well plates (18 hr. 370 C, 5% CO2) in fresh medium under the following conditions: a) RPMI control; b) NS-398 (10-100 μM) for 18 hours; c) CGS-21680 10-5M for 18 hours; d) NS-398 50 μM for 18 hr+CGS-21680 10-5M for 18 hours.

27-OHase and ABCA1 mRNA were quantitated by real-time PCR. cDNA was copied from 5 μg of total RNA using M-MLV reverse transcriptase primed with oligo dT. Equal amounts of cDNA were taken from each RT reaction mixture for PCR amplification using cholesterol 27-OHase-specific primers or ABCA1 specific primers as well as glyceraldehyde-3-phosphate dehydrogenase (GAPDH) control primers. The cholesterol 27-OHase-specific primers span a 311 base-pair sequence encompassing nucleotides 491-802 of the human cholesterol 27-OHase cDNA. ABCA1 primers yield a 234 BP amplified fragment. Real-time PCR analysis was performed using the SYBR Green PCR Reagents Kit (Applied Biosystems) with a Stratagene MX3005P QPCR System according to manufacturer's instructions.

Total cell lysate was isolated for Western immunoblotting using RIPA lysis buffer (98% PBS, 1% Igepal CA-630, 0.5% sodium deoxycholate, 0.1% SDS). 100 μl of RIPA lysis buffer and 10 μl of protease inhibitor cocktail (Sigma) was added to the cell pellet from each condition, incubated on ice for 35 minutes and vortexed every 5 minutes. Supernatant was collected after centrifuging at 10,000 g at 4° C. for 10 minutes using an Eppendorf 5415C centrifuge. The quantity of protein in each supernatant was measured by absorption at 560 nm using a Hitachi U2010 spectrophotometer.

As a control, on the same transferred membrane, beta-actin was detected using mouse anti-beta-actin (diluted in 1:1000, from abCam, product Code: ab6276) and ECL sheep anti-mouse-IgG horseradish peroxidase-linked species-specific whole antibody (diluted in 1:2000, from Amersham Biosciences, product Code NA931) and all other similar steps as above.

Statistical analysis was performed using SigmaStat v2.03 (SPSS, Inc., Chicago, Ill.). Data was analyzed using the Kruskal-Wallis One-Way Analysis of Variance on Ranks. Pairwise multiple comparison was made with the Holm-Sidak method.

Additional Experiments: Foam Cell Formation

THP-1 human monocytes (1×10⁶ cells/ml) in 12 well plates were treated with phorbol dibutyrate, 300 nM (Sigma) for 48 hr at 37° C. to facilitate differentiation into macrophages. The differentiated macrophages were washed three times with PBS, then incubated alone or in the presence of 10 μM NS-398 (37° C., 5% CO2, 18 hours). Cells were cholesterol-loaded with acetylated LDL (50 μg/ml) and further incubated in RPMI (37° C., 5% CO2) for 48 hours. Studies were performed in triplicate.

Immediately following incubation, media was aspirated and cells were fixed in the same 12 well plates used for incubation, with 4% paraformaldehyde in water, for 2-4 minutes. Cells were stained with 0.2% Oil-Red-O in methanol for 1-3 min. Cells were observed via light microscope (Axiovert 25-Zeiss) with 100× magnification and then photographed using a Kodak DC 290 Zoom Digital Camera. The number of foam cells formed in each condition were calculated manually and presented as percentage foam cell formation.

TUP-1 monocytoid cells (10⁶ cells/ml) were incubated (18 hrs, 37° C., 5% CO2) with/without the selective COX-2 inhibitor NS-398 (50 μM) in the presence or absence of the A2AR specific agonist CGS-21680 (10 μM). RNA were collected directly from the culture dishes using the Trizol reagent and subjected to quantitative real-time PCR for 27-OHase and ABCA1 using 5 μg of total RNA per condition for reverse transcription with oligo dT primers.

In cultured TUP-1 human monocytes, NS-398 greatly reduced expression of mRNA for the cholesterol-metabolizing 27-OHase enzyme in a dose-dependent manner. Message level of the cholesterol efflux protein ABCA1 is also reduced following NS-398 exposure. Results were confirmed by immunoblot. Addition of an anti-inflammatory adenosine A2AR agonist overcame the reduction in both 27-OHase and ABCA1. Addition of CGS-21680 to NS-398-treated THP-1 cells gave rise to a 184% increase in 27-OHase and a 141% increase in ABCA1 expression (for 27-OHase: 167.2±8.57% in CGS-21680+NS-398 versus 58.9±2.3% inNS-398 alone, n=3, p<0.001, and for ABCA1: 146.0±3.15% in CGS-21680+NS-398 versus 60.49±4.42% in NS-398 alone, n=3, p<0.001. 100%=baseline expression in untreated THP-1 cells).

Discussion of Results

Increased cardiovascular risk with COX-2 inhibition may be ascribed at least in part to disruption of cholesterol outflow which can be corrected via activation of a specific adenosine receptor. These findings provide a novel therapeutic approach targeting the A2AR to decrease the cardiovascular consequences of COX-2 inhibitor therapy in those who require chronic analgesia for arthritic and other inflammatory conditions.

This is the first evidence that any widely used pharmacotherapy can increase the expression of the anti-atherogenic 27-OHase or ABCA1 and can counteract the effects of COX-2 inhibition or IFN-γ exposure on gene expression. It was demonstrated that MTX inhibits foam cell formation under conditions of lipid overload. The capacity of MTX to reduce the burden of ASCVD in patients with RA may be ascribed, in part, to favorable alterations in cholesterol homeostasis mediated via activation of the adenosine A2A receptor. Thus, adenosine receptor ligation provides a suitable mechanism for a promising treatment paradigm with long term-benefit in ASCVD.

In order to extend showings of the effects of the A2A agonists on cholesterol metabolism disruption by commercial COX-2 inhibitors, experiments were separately specifically conducted with the COX-2 inhibitor, celecoxib.

CELECOXIB Example and Testing

Celecoxib remains the only available COX-2 inhibitor for treatment of arthritis pain and inflammation in the Unites States. Valdecoxib and Rofecoxib have been withdrawn from the market due to increased evidence of cardiovascular risk.

The exact mechanisms by which COX-2 inhibition may impart increased atherogenic effects has not been elucidated, and concern remains the risk may be a class effect. We showed that selective inhibition of COX-2 with NS-398 downregulates reverse cholesterol transport protein ABCA1, a key regulator involved in ridding cells of cholesterol accumulation. As with other COX-2 inhibitors, Celecoxib exhibited atheroma-promoting properties via compromise of RCT in TUP-1 macrophages and imparted increased vulnerability to foam cell formation. COX-2 gene silencing appears to adversely affect RCT since both celecoxib and NS-398 suppress RCT through COX-2 inhibition.

Methods: TUP-1 monocytes (RPMI 1640, 37° C., 5% CO2) were grown to a density of 10⁶ cells/ml. TUP-1 cells were then either subjected to the experimental conditions described or differentiated into adherent macrophages (phorbol dibutyrate, 300 nM, 48 h).

Media was aspirated and cells were rinsed 3 times with Dulbecco's Phosphate Buffered Saline (PBS) without Ca++ and Mg++. The cells were incubated in 6-well plates under the following conditions in triplicate: (1) media alone; (2) 10 μM NS-398 (18 h); (3) 50 μM NS-398 (18 h); (4) 10 μM celecoxib (18 h); (5) 50 μM celecoxib (18 h); (6) 500 U/ml IFN-γ (12 h); (7) 50 nM siRNA (24 h); (8) 50 nM mock siRNA (24 h). THP-1 macrophages were cholesterol-loaded with 50 μg/ml acetylated LDL and incubated an additional 48 h prior to oil red O staining.

Cells were collected and centrifuged at 1500 RPM at room temperature, media was aspirated and, cell protein and RNA were isolated (Trizol reagent). The quantity of total RNA from each condition was measured by absorption at 260 nanometer wavelengths using quartz cuvettes by ultraviolet (UV) spectrophotometry (Beckman Coulter DU800).

RT and PCR reactions were carried out in an Eppendorf master cycler-personal. For each assay 1 μg of mRNA was reverse transcribed using Omniscript reverse transcriptase in the presence of 10 U of RNase inhibitor and 1 μM oligodT primers. Equal amounts of cDNA were taken from each RT reaction mixture for PCR amplification using both ABCA1 specific primers and glyceraldehyde-3-phosphate dehydrogenase (GAPDH) control primers. ABCA1 primers yield a 234 bp, GAPDH primers a 357 bp, amplified fragments. The PCR products were loaded directly onto a 2% agarose gel and electrophoresed at 5 V/cm for 1.5 hr. DNA was ethidium bromide stained, then visualized and photographed under UV light with a Kodak trans-illuminator. The gel images were photo documented and net intensities were measured with Kodak digital science 1D, version 2.0.3, after imaging with Kodak Digital Science Electrophoresis Documentation and Analysis System 120. All experimental results were normalized to the mean density of GAPDH.

Cell transfection and COX silencing: Cells were washed 3 times in PBS. Fresh RPMI 1640 (100 ml) was added to each appropriate 8-chamber plate. Cells were incubated in the short time prior to transfection. The following is described per chamber well for a 24 well plate. 375 ng of siRNA (PTGS2_{—}5 HP Validated siRNA, Qiagen) was diluted in 100 ml culture medium without serum (final concentration of 50 nM). Addition of 6 ml of HiPerFect Transfection Reagent to the diluted siRNA was followed by vortex mixing. The samples were incubated 5-10 min at room temperature to allow formation of transfection complexes. The complexes were added drop wise onto the cells and swirled to ensure uniform distribution. The cells were then incubated for 6 hr under normal growth conditions. Then 400 ml culture medium containing serum and antibiotics were added, and cells were further incubated for 24-72 hours.

Following incubation, media was aspirated and cells were fixed with 4% paraformaldehyde in water, for 2-4 min. Cells were stained with 0.4% trypan blue for 1-3 min, washed in PBS, and then stained with 0.2% oil-Red-O in methanol for 1-3 min. Observation and photography of cells was performed respectively via light microscope (NIKON Eclipse TE300) with 40× magnification and SONY Progressive 3CCD color video camera. The number of foam cells formed in each condition were calculated manually and presented as percentage foam cell formation.

Western Blots: Total cell lysate protein samples (10 μg/lane) were boiled for 5 min, loaded onto a 7.5% polyacrylamide gel, electrophoresed for 1.5 hr at 100V then transferred to a nitrocellulose membrane in a semi-dry transblot apparatus for 1 hr at 100V. For ABCA1 detection, the primary antibody was a rabbit anti-human ABCA1 antibody (1:2000 dilution, Santa Cruz Biotechnology). Secondary antibody was ECL donkey anti-rabbit IgG Horseradish peroxidase-linked species-specific whole antibody (1:4000 dilution).

Statistical analysis was performed using Graphpad prism, version 5.01 and SigmaStat version 2.03. Pairwise multiple comparison was made between control and treatment conditions using unpaired t tests, two tailed 95% confidence intervals, significance p<0.05. Foam cell formation was analyzed by Kruskal-Wallis one-way analysis of variance on ranks and pairwise multiple comparison was made with the Holm-Sidak method.

Results: Celecoxib significantly decreased ABCA1 message, 50 μM resulted in 65.1%±1.5 decrease vs. control, p=0.003. Celecoxib induced ABCA1 protein expression decreased in a dose dependent manner, p=0.0002. Transfection with COX-2 siRNA significantly diminished ABCA1 message, 22.9%±4.9 decrease vs. mock, p=0.03.

As shown in FIGS. 1a and 1b, Celecoxib decreased ABCA1 protein expression, 50 μM resulted in decrease of 25.6%±1.3 vs. control, p<0.0001. Lipid laden foam cell formation increased with 50 μM Celecoxib, resulting in 95.0%±0.7 foam cells vs 39.1%±5.4 control, p=0.003.

TUP-1 macrophages transfected with COX-2 gene silencer had greater propensity to form lipid laden foam cells than mock transfected cells, 58.3%±1.6 increase, p=0.003.

Photomicrographs were taken of cholesterol loading with acetylated LDL alone then with 50 μM Celebrex treated cells followed by cholesterol loading with acetylated LDL. This was followed by cholesterol loading with acetylated LDL after mock transfection COX-2 siRNA treated cells followed by cholesterol loading with acetylated LDL.

MTX and CGS-21680 Test Results

MTX (5 μM, 18 hr) increased 27-OHase mRNA expression (113.9±6.4%) and completely blocked NS-398-induced downregulation of 27-OHase message (112.8±13.1% for NS-398+MTX versus 71.1±4.3% for NS-398 alone, with untreated as 100%, n=3, p<0.01) (shown in FIG. 3d). This ability of MTX to overcome suppression of 27-OHase expression by NS-398 was observed at MTX doses of 0.1 μM, 0.5 μM and 5 μM at both the protein and message level (shown in FIG. 5).

MTX was also effective in blocking COX-2 inhibitor-mediated downregulation of ABCA1 message in TUP-1 monocytes. Adenosine A2A receptor blockade with ZM-241385 abolished the ability of MTX to counter COX-2 inhibitor effects on both 27-OHase (shown in FIGS. 3c and 4b) and ABCA1. Similarly, downregulation of 27-OHase and ABCA1 by IFN-γ in TUP-1 monocytes, which we showed previously, was also prevented by MTX and this effect of MTX was negated by ZM-241385 (shown in FIGS. 6a and 6b).

Addition of the adenosine A2A receptor agonist CGS-21680 to TUP-1 monocytes exposed to NS-398 overcame the reduction in 27-OHase expression. This was demonstrated by immunoblot and QRT-PCR. Addition of CGS-21680 to NS-398-treated THP-1 cells gave rise to a 184% increase in 27-OHase mRNA (167.2±8.57% in CGS+NS-398 vs. 58.9±2.3% in NS-398 alone, n=3, p<0.001).

MTX on foam cell levels: Acetylated LDL-treated TUP-1 macrophages showed a significant decrease in foam cell transformation in the presence of MTX compared to control (29.7×2.0% vs. 39.3±5.0%, p<0.001). NS-398 treatment resulted in 72.7±4.9% foam cells while combined NS-398+MTX resulted in only 36.3±3.2% foam cells, (n=3, p<0.001). IFN-γ treatment prior to cholesterol loading with acetylated LDL resulted in 71.0±5.0% foam cells while IFN-γ+MTX resulted in only 46.0±7.2% foam cells, (n=3, p<0.001). Preincubation of THP-1 macrophages with the selective A2A receptor antagonist (ZM-241385) prior to MTX treatment ablated the anti-atherogenic effect of MTX and resulted in a significant increase in foam cells (62.1±1.5%).

COX-2 inhibitor-mediated decrease in 27-OHase mRNA is prevented by MTX. TUP-1 human monocytes were exposed to the following conditions represented by the four bars (from left to right, FIG. 3d): (1) control RPMI 1640, (2) MTX (5 μM, 18 hr), (3) NS-398 (50 μM, 18 hr), (4) MTX (5 μM, 18 hr) and NS-398 (50 μM, 18 hr). Cells were extracted for total RNA, and evaluated for 27-OHase mRNA expression by QRT-PCR. Signals obtained from the amplification of GAPDH message were used as internal controls. *p<0.05, control vs. NS-398. #p<0.01, NS-398+MTX vs. NS-398 (the *p and #p notations refer to the p values in the appropriate bar graphs to indicate which p value fits which bar).

Detection and quantitation of cholesterol 27-OHase in NS-398-treated TUP-1 cells exposed to increasing doses of MTX was examined, revealing a decrease in 27-OHase protein in TUP-1 monocytes treated with the COX-2 inhibitor NS-398 that is corrected with increasing concentrations of MTX. Cultured TUP-1 monocytic cells were untreated or exposed to NS-398 (50 μM, 18 hr) then untreated or exposed to increasing doses of MTX for 24 hr. Total cell protein was isolated and 27-OHase detected with specific rabbit polyclonal anti-human 27-OHase antibody. Western blotting was also performed with an anti-beta actin antibody to confirm equal protein loading.

COX-2-inhibitor-mediated suppression of27-OHase MRNA expression in TUP-1 monocytes is overcome with MTX. Cultured TUP-1 monocytic cells were incubated in NS-398 (50 μM, 48 hr) then untreated or exposed to increasing doses of MTX for 24 hr. Following isolation of total RNA, the RNA was reverse transcribed and the cDNA amplified by QRT-PCR as described. Signals obtained from the amplification of GAPDH message were used as internal controls. *p<0.05, **p<0.01, MTX vs Control (C). #p<0.01, NS+MTX vs NS-398 (NS).

Detection and quantitation of cholesterol 27-OHase and ABCA1 mRNA in NS-398-treated THP-1 cells exposed to MTX in the presence and absence of A2A receptor antagonism with ZM-241385 demonstrates that suppression of 27-OHase message in THP-1 cells by NS-398 is reversed by MTX and this reversal is blocked by ZM-241385. TUP-1 monocytes were exposed to the following conditions represented by the four bars from left to right (FIG. 3c): (1) Control RPMI 1640, (2) NS-398 (50 μM, 24 hr), (3) NS-398 (50 μM, 24 hr) then add MTX (5 μM, 24 hr), (4) NS-398 (50 μM) and ZM-241385 (10 μM) for 24 hr. then add MTX (5 μM) for 24 hr. Cells were extracted for total RNA, and evaluated for 27-OHase mRNA by QRT-PCR. Signals obtained from the amplification of GAPDH message were used as internal controls.

It is further demonstrated that suppression of ABCA1 message in TUP-1 cells by NS-398 is reversed by MTX and this reversal is blocked by ZM-241385. TUP-1 monocytes were exposed to the following conditions represented by the four bars from left to right (FIG. 5): (1) Control RPMI 1640, (2) NS-398 (50 μM, 24 hr), (3) NS-398 (50 μM, 24 hr) then add MTX (5 μM, 24 hr), (4) NS-398 (50 μM) and ZM-241385 (10 μM) for 24 hr. then add MTX (5 μM) for 24 hr. Cells were extracted for total RNA, and evaluated for 27-OHase mRNA by QRT-PCR. Signals obtained from the amplification of GAPDH message were used as internal controls. *p<0.05, **p<0.01, MTX vs control (C). #p<0.01, NS-398+MTX vs NS-398.

Detection and quantitation of cholesterol 27-OHase mRNA and protein and ABCA1 mRNA in IFN-γ-stimulated TUP-1 cells exposed to MTX in the presence and absence of A2A receptor antagonism with ZM-241385 demonstrated suppression of 27-OHase message in THP-1 cells by IFN-γ is reversed by MTX and this reversal is blocked by ZM-241385. THP-1 monocytes were exposed to the following conditions represented by the eight bars from left to right (FIGS. 6a and 6b): (1) Control RPMI 1640, (2) ZM-241385 (10 μM, 24 hr), (3) MTX (5 μM, 24 hr), (4) IFN-γ (500 U/ml, 24 hr), (5) IFN-γ (500 U/ml, 24 hr), then add MTX (5 μM, 24 hr), (6) ZM-241385 (10 μM, 24 hr), then add MTX (5 μM, 24 hr), (7) ZM-241385 (10 μM) and IFN-γ (500 U/ml) for 24 hr. then add MTX (5 μM, 24 hr), (8) ZM-241385 (10 μM) and NS-398 (50 μM) for 24 hr. then add MTX (5 μM, 24 hr). Cells were extracted for total RNA, and evaluated for 27-OHase mRNA by QRT-PCR. Signals obtained from the amplification of GAPDH message were used as internal controls.

It was further demonstrated that suppression of 27-OHase protein in THP-1 cells by IFN-γ is reversed by MTX and this reversal is blocked by ZM-241385. THP-1 monocytes were exposed to identical conditions 1-8 as in part (a) of FIG. 6A-6B represented by the eight lanes of the immunoblot from left to right. Total cell protein was isolated and 27-OHase detected with specific rabbit polyclonal anti-human 27-OHase antibody. Western blotting was also performed with an anti-beta actin antibody to confirm equal protein loading.

Additionally, it was found that suppression of ABCA1 message in TUP-1 cells by IFN-γ is reversed by MTX and this reversal is blocked by ZM-241385. THP-1 monocytes were exposed to identical conditions 1-8 as in part (a) of FIG. 6A, represented by the eight bars from left to right. Cells were extracted for total RNA, and evaluated for 27-OHase mRNA by QRT-PCR. Signals obtained from the amplification of GAPDH message were used as internal controls.

CGS-21680: The effect of the A2A agonist CGS-21680 on NS-398-induced suppression of 27-OHase expression in TUP-1 monocytes was examined, revealing that 27-OHase message level is decreased by the COX-2 inhibitor, NS-398 (50 μM), and this decrease is reversed by the addition of the adenosine A2A agonist, CGS-21680 (10 μM). TUP-1 monocytes were exposed to the following conditions represented by the four bars from left to right (FIG. 3a): (1) Control RPMI 1640, (2) CGS-21680 (10 μM, 18 hr), (3) NS-398 (50 μM, 18 hr), (4) NS-398 (50 μM, 18 hr) and CGS-21680 (10 μM, 18 hr). Cells were extracted for total RNA, and evaluated for 27-OHase mRNA by QRT-PCR. Signals obtained from the amplification of GAPDH message were used as internal controls. *p<0.01, control vs. NS-398. #p<0.01, NS-398+CGS-21680 vs. NS-398.

In a further study, THP-1 monocytes were exposed to the following conditions: (1) Control RPMI 1640, (2) CGS-21680 (10 μM, 18 hr), (3) NS-398 (50 μM, 18 hr), (4) NS-398 (50 μM, 18 hr) and CGS-21680 (10 μM, 18 hr) and evaluated by immunoblot for expression of 27-Ohase protein. This study demonstrated significant beneficial results. *p<0.01, control vs. NS-398. #p<0.01, NS-398+CGS-21680 vs. NS-398, and this decrease is reversed by the addition of the adenosine A2A agonist CGS-21680. It has been thus established that 27-OHase protein level is decreased by the COX-2 inhibitor, NS-398, and this decrease is reversed by the addition of the adenosine A2A agonist CGS-21680.

MTX on NS-398 and IFN-γ-induced foam cell transformation in lipid loaded THP-1 macrophages: Representative photomicrographs were taken at 40× magnification of lipid laden macrophages stained with oil red-O. Findings include that: (a) acetylated LDL-treated TUP-1 macrophages showed a significant decrease in foam cell transformation in the presence of MTX compared to control; (b) MTX prevented the NS-398-induced increase in foam cell formation in TUP-1 macrophages; (c) MTX prevented the IFN-γ-induced increase in foam cell formation in TUP-1 macrophages; and (d) effectiveness of MTX in decreasing foam cell formation is abolished by A2AR antagonism with ZM-241385.

NS-398 dramatically reduced cholesterol 27-OHase message in TUP-1 cells in a dose-dependent manner. This result was confirmed by Western immunoblot. 27-OHase protein expression decreased in the presence of the COX-2 inhibitor NS-398.

ABCA1 message was reduced following NS-398 exposure to approximately 70% of control (50 μM, 71.1±3.9% of control, n=3, p<0.01). This result was confirmed by Western immunoblot.

Addition of the adenosine A2A receptor agonist CGS-21680 overcame the reduction in both 27-OHase and ABCA1 expression (FIG. 3a). Addition of CGS-21680 to NS-398-treated TUP-1 cells gave rise to a 184% increase in 27-OHase mRNA and a 141% increase in ABCA1 mRNA (for 27-OHase: 167.2±8.57% in CGS-21680+NS-398 vs 58.9±2.3% in NS-398 alone, n=3, p<0.001, and for ABCA1: 146.0±3.15% in CGS-21680+NS-398 vs 60.49±4.42% inNS-398 alone, n=3, p<0.001). 100%=baseline expression in untreated THP-1 cells.

THP-1 macrophages treated with the selective COX-2 inhibitor NS-398 showed greater vulnerability to form lipid-laden foam cells compared to untreated cells under conditions of cholesterol-loading with acetylated LDL. THP-1 macrophages showed a significant increase in foam cell transformation in the presence of NS-398 compared to control (42.7±6.6 vs 20.1±3.4%, p=0.04).

THP-1 monocytoid cells (10⁶ cells/ml) were incubated (18 hrs, 37° C., 5% CO2) with/without the selective COX-2 inhibitor NS-398 (50 μM) in the presence or absence of the A2AR specific agonist CGS-21680 (10 μM). RNA were collected directly from the culture dishes using the Trizol reagent and subjected to quantitative real-time PCR for 27-OHase and ABCA1 using 5 μg of total RNA per condition for reverse transcription with oligo dT primers.

In summary, it has been found that in cultured TUP-1 human monocytes, NS-398 greatly reduced expression of mRNA for the cholesterol-metabolizing 27-OHase enzyme in a dose-dependent manner. Message level of the cholesterol efflux protein ABCA 1 is also reduced following NS-398 exposure. Results were confirmed by immunoblot. Addition of an anti-inflammatory adenosine A2AR agonist overcame the reduction in both 27-OHase and ABCA1. Addition of CGS21680 to NS-398-treated TUP-1 cells gave rise to a 184% increase in 27-OHase and a 141% increase in ABCA1 expression (for 27-OHase: 167.2±8.57% in CGS-21680+NS-398 versus 58.9±2.3% in NS-398 alone, n=3, p<0.001, and for ABCA 1: 146.0±3.15% in CGS-21680+NS-398 versus 60.49±4.42% in NS-398 alone, n=3, p<0.001. 100%=baseline expression in untreated THP-1 cells).

It is understood that the above examples are illustrative of the present invention and that different COX-2 inhibitors or other NSAIDS which disrupt the cholesterol metabolic function, as well as the A2A agonists which restore the function are similarly within the scope of the present invention. It is contemplated that treatments using the present invention could be implemented using any of several routes of administration, including but not limited to central, systemic, peripheral, intravenous, subcutaneous, oral, nasal and/or transdermal routes of admnistration. Effective routes of administration would thus be readily determined and practiced by those of average skill in the art. Dosage amounts whether absolute or relative as well as times for dosing and the like are similarly variable depending on conditions and patient susceptibility to risk and the like in accordance with the following claims. Proper dosages and treatment regimens would readily be understood and put into practice by one of skill in the art. It is also understood that A2A agonists could be administered in conjunction with, or seperately from, COX-2 inhibitors, in either a simultaneous or sequential fashion.

Although the present invention has been described in relation to particular embodiments thereof, many other variations and modifications and other uses will readily be apparent to those skilled in the art. It is understood, therefore, that the present invention is not limited by the specific disclosure herein.

What is claimed is:

1. A method of reducing the increased risk of adverse atherosclerotic cardiovascular and cerebrovascular conditions in a human resulting from the use by the human of a COX or selective COX-2 inhibitor, the method comprising:
restoring disrupted cholesterol metabolic function engendered by a COX or selective COX-2 inhibitor, and
reducing the production of lipid laden foam cells caused by the use of the COX or COX-2 inhibitor.

2. The method of claim 1, wherein the disrupted cholesterol metabolic function is restored and the production of lipid laden foam cells is reduced by administering an adenosine A2A receptor agonist having a threshold level of activity of about 0.1 μM, to the human using the COX or COX-2 inhibitor.

3. The method of claim 2, wherein the adenosine A2A receptor agonist is administered to the human in amounts sufficient to substantially only saturate the A2A receptor.

4. The method of claim 2, wherein the adenosine A2A receptor agonist is administered to the human at time intervals sufficient to maintain the restored cholesterol metabolic function during the time that the COX or COX-2 inhibitor is being used.

5. The method of claim 2, wherein the adenosine A2A receptor agonist is selected from the group consisting of methotrexate, CGS-21680; ATL 313; Binodenoson; MRE-0094; UK-371,104; Regadenoson; Apadenoson; APEC; and 2HE-NECA.

6. The method of claim 2, wherein the inhibitor is a selective COX-2 inhibitor.

7. The method of claim 6 wherein the selective COX-2 inhibitor is selected from the group consisting of celecoxib, valdecoxib and rofecoxib.

8. The method of claim 5, wherein the COX-2 inhibitor is selected from the group consisting of celecoxib, valdecoxib and rofecoxib.

9. The method of claim 2, wherein the A2A receptor agonist is specific to the A2A receptor.

10. The method of claim 2, wherein the A2A receptor agonist is administered to the human in an oral dose.

11. A pharmaceutical composition comprising a pharmaceutically acceptable selective COX-2 inhibitor and a pharmaceutically acceptable A2A receptor agonist, with the A2A receptor agonist being present in an amount sufficient to restore cholesterol metabolic function disrupted by the selective COX-2 inhibitor.

12. The method of claim 2, wherein the inhibitor is a COX inhibitor.

13. The pharmaceutical composition of claim 10, wherein the pharmaceutical composition is formulated for oral administration.

14. A method of reducing the risk or myocardial infarction and stroke in a human to whom COX or selective COX-2 inhibitors are administered, comprising administering to said human an adenosine A2A receptor antagonist.

15. A method of ameliorating atherogenesis in a mammal including a human, comprising: administration of an adenosine A2A receptor agonist to mammal in need thereof in an amount substantially only sufficient to saturate the A2A receptor of the human, wherein atherogenesis in the subject is thereby reduced or prevented.

16. The method of claim 15, wherein the adenosine A2A receptor agonist is selected from the group consisting of methotrexate, CGS-21680, ATL 313, Binodenoson, MRE-0094, UK-371,104, Regadenoson, Apadenoson, APEC and 2HE-NECA.

17. The method of claim 15, wherein the adenosine A2A receptor agonist is administered with sufficient frequency to maintain the human's cholesterol metabolic function.

18. The method of claim 15, wherein the administration is oral.

19. The method of claim 15, further wherein the human is administered an effective therapeutic amount of a selective COX-2 inhibitor.

20. The method of claim 19, wherein the administration of the selective COX-2 inhibitor is simultaneous or sequential with the administration of the adenosine A2A receptor agonist.

21. The method of claim 19, wherein the administration of the selective COX-2 inhibitor with the administration of the adenosine A2A receptor agonist is with sufficient frequency to maintain the human's cholesterol metabolic function.

22. The method of claim 19, wherein the administration of the selective COX-2 inhibitor and the adenosine A2A receptor agonist are oral.

23. The method of claim 19, wherein the selective COX-2 inhibitor is selected from the group consisting of celecoxib, valdecoxib and rofecoxib.

24. A pharmaceutical composition comprising: a pharmaceutically acceptable selective COX-2 inhibitor and a pharmaceutically acceptable A2A receptor agonist, with the A2A receptor agonist being present in an amount sufficient to reduce or prevent atherogenesis in a subject.

25. The pharmaceutical composition of claim 24, wherein the pharmaceutical composition is formulated for oral administration.

26. A method of reducing effect in a mammal resulting from the use by the mamal of a COX or selective COX-2 inhibitor which disrupts a normal cholesterol metabolic function; the method comprising restoring disrupted cholesterol metabolic function engendered by COX or selective COX-2 inhibitor.

27. A method of reducing the increased risk of adverse atherosclerotic cardiovascular and cerebrovascular conditions in a human resulting from the use by the human of a COX or selective COX-2 inhibitor, the method comprising restoring disrupted cholesterol metabolic function engendered by COX or selective COX-2 inhibitor.

28. The method of claim 15 wherein the mammal is a human.

29. The method of claim 1, wherein the COX or selective COX-2 inhibitor disrupts a normal cholesterol metabolic function and causes production of lipid laden foam cells.
